(19) United States
(12) Patent Application Publication
(10) Pub. No.: US 2023/0310513 A1
(43) Pub. Date: Oct. 5, 2023

(54) METHOD FOR FEEDING A FLATWORM AND FLATWORM EXTRACT FREE OF HUMAN-PATHOGENIC MICROORGANISMS

(71) Applicants: Eric GHIGO, Marseille (FR); FONDATION MEDITERRANEE INFECTION, Marseille (FR); AIX-MARSEILLE UNIVERSITE, Marseille Cedex 07 (FR)

(72) Inventors: Didier RAOULT, Marseille (FR); Pierre Edouard FOURNIER, Marseille (FR); Eric GHIGO, Marseille (FR); Eric CHABRIÈRE, Aubagne (FR)

(73) Assignees: Eric GHIGO, Marseille (FR); FONDATION MEDITERRANEE INFECTION, Marseille (FR); AIX-MARSEILLE UNIVERSITE, Marseille Cedex 07 (FR)

(21) Appl. No.: 18/024,682

(22) PCT Filed: Aug. 30, 2021
(86) PCT No.: PCT/FR2021/051497

§ 371 Date: Mar. 3, 2023

(30) Foreign Application Priority Data
Sep. 4, 2020 (FR) FR2008979

(51) Int. Cl. A61K 35/62; A61P 17/02; A01K 67/033

(52) U.S. Cl. CPC A61K 35/62; A61P 17/02; A01K 67/0335; A01K 2227/703; A01K 2207/25; A01K 2267/01

(57) ABSTRACT

The invention relates to a method for feeding a flatworm and to a flatworm extract that is free of human-pathogenic microorganisms and is useful in the pharmaceutical, cosmetic and nutraceutical fields.

### TECHNICAL FIELD

The present invention relates to a method for feeding a flatworm and to a flatworm extract that is free of human-pathogenic microorganisms, useful in the pharmaceutical, cosmetic and nutraceutical fields.

### PRIOR ART

The term “flatworms” designates a phylum of tapeworms which includes four classes: the rhabditophora (formerly “turbellarians”) such as the planarian, the monogeneans which are parasites of aquatic organisms, the trematodes (flukes, digeneans) which are parasites such as liver fluke, and cestodes such as tape worm or taenia.

Planarians are aquatic tapeworms belonging to several species in the class of Rhabditophora. They can be swimmers or crawlers, and live at sea, in fresh water, or in very humid soils (in tropical forests). Planarians are known for their regenerative abilities. They are able to perpetually regenerate themselves, and any part of their body, due to the presence of a large amount of stem cells (20-30%) in their tissues compared to other living organisms for which the amount of stem cells is of the order of 1 ppm. Planarian stem cells multiply and regenerate continuously, unlike the stem cells of more evolved organisms, which multiply little or not at all. The biochemical factors allowing the multiplication of stem cells in a planarian remain unidentified.

Flatworms are known to feed on substances of animal origin in their natural environment, and are generally fed with calf liver or chicken liver when reared in the laboratory. Because of their diet, flatworms harbor human-pathogenic microorganisms in their microbiota.

Flatworm extracts are described in the literature as having numerous pharmacological properties.

Patent application WO2019016346 describes a planarian aqueous extract, which is non-parasitic and non-pathogenic in humans, in particular of the Schmidtea mediterranea species, capable of activating the multiplication of human stem cells and which therefore has a cell regeneration activity useful in numerous cosmetic, pharmaceutical or nutraceutical applications.

The flatworms used to obtain flatworm extracts are generally fed with calf liver. However, the analysis of their microbiota revealed the presence of human-pathogenic microorganisms, such as Aeromonas veronii, Staphylococcus capitis, Pseudomonas fluorescens, Acinetobacter guillouiae, Aeromonas hydrophila, Delftia acidovorans, Comamonas testosteroni, Sphingomonas paucimobilis, Staphylococcus epidermidis, Micrococcus luteus and/or Staphylococcus haemolyticus.

These microorganisms can be problematic in all cosmetic, pharmaceutical or nutraceutical applications resulting from the use of flatworm extracts or flatworms as such.

### SUMMARY OF THE INVENTION

In the context of the present invention, it has been observed that it was very difficult to be able to sustainably feed flatworms with a food free of element of animal origin. Fortuitously, the Applicant was able to observe that flatworms agree to feed with malted yeast, which is a food free of any element of animal origin.

In the context of the present invention, it has also been shown quite fortuitously that it was possible to have flatworms free of human-pathogenic microorganisms by feeding said flatworms with malted yeast. The extracts obtained from these “healthy” flatworms retain the ability to activate the multiplication of human stem cells and therefore have a cell regeneration activity, in particular stem or differentiated cells.

A first object of the present invention relates to a method for feeding flatworms, in which the flatworms are fed exclusively with malted yeast.

A second object of the present invention relates to a method for preparing a flatworm extract, comprising the steps of:
(i) feeding flatworms by implementing the feeding method according to the first object of the invention, and
(ii) preparing a flatworm extract from the flatworms obtained in step (i).

A third object of the present invention relates to a flatworm extract fed exclusively with malted yeast capable of being obtained by implementing the method according to the second object of the invention, said flatworms being free of human-pathogenic microorganisms.

A fourth object of the present invention relates to a composition comprising one or more flatworms fed exclusively with malted yeast, said flatworm(s) being free of human-pathogenic microorganisms.

A fifth object of the present invention relates to a composition comprising an extract according to the invention.

A sixth object of the present invention relates to a method for eliminating pathogenic microorganisms present in one or more flatworms, said method comprising a step which consists in feeding one or more flatworms, preferably exclusively, with malted yeast for a sufficient period to eliminate said pathogenic microorganisms.

A seventh object of the present invention relates to a method for preparing a flatworm extract according to the invention, comprising the steps of: (a) obtaining flatworms free of human-pathogenic microorganisms by implementing the method for eliminating pathogenic microorganisms from flatworms according to the invention; (b) preparing a flatworm extract from the flatworms obtained in step (a).

An eighth object of the present invention relates to the use of an aqueous extract according to the invention or of a composition comprising an extract according to the invention, for cultivating human or animal cells in vitro or ex vivo.

A ninth object of the present invention relates to an extract according to the invention or a composition comprising an extract according to the invention, for the use thereof as a medicament, for example to be used to promote the healing of a wound in the skin or a skin burn.

### DETAILED DESCRIPTION

### Definitions

The term “flatworm” refers to a flatworm from the phylum of the same name. In the context of the invention, the flatworm can be of the class of rhabditophora, preferably it is a planarian. Preferably, the flatworm according to the invention is non-parasitic and/or non-pathogenic in humans. Advantageously, the flatworm according to the invention is a planarian belonging to one of the following species Schmidtea mediterranea, Dugesia japonica, Dendricoelum lacteum, Polycelis nigra, Polycelys tenuis and planarian torva, preferably to Schmidtea mediterranea species.

“Malted yeast” or “brewer's yeast” is well described in the literature, it is the result of growing Saccharomyces cerevisiae with malt, usually barley malt. It usually comes in the form of flakes and is widely used in vegetarian diets. The malted yeast according to the present invention can be in any form suitable for feeding flatworms, for example in the form of powder, flakes, granules or in liquid form, for example obtained by mixing flakes with water. The yeasts present in the malted yeast are said to be “active”, that is to say they are alive.

The expression “flatworm free of human-pathogenic microorganisms” designates a flatworm whose digestive tract is free of human-pathogenic microorganisms, that is to say a flatworm whose microbiota is free of human-pathogenic microorganisms. In a particular embodiment, the human-pathogenic microorganisms are Aeromonas veronii, Staphylococcus capitis, Pseudomonas fluorescens, Acinetobacter guillouiae, Aeromonas hydrophila, Delftia acidovorans, Comamonas testosteroni, Sphingomonas paucimobilis, Staphylococcus epidermidis, Micrococcus luteus and Staphylococcus haemolyticus. In this particular embodiment, the microbiota of the flatworm of the invention is therefore free of the set of pathogenic microorganisms mentioned above.

The term “extract” designates the product resulting from the extraction of the contents of the cells. Thus, the term “extract of one or more flatworms” designates the product resulting from the extraction of the contents of the cells of said flatworm(s).

The term “pharmaceutically acceptable” means approved by a federal or state regulatory agency or listed in the United States or European Pharmacopoeia, or other generally recognized pharmacopoeia, for use in animals and humans. A “pharmaceutical composition” means a composition comprising a pharmaceutically acceptable vehicle. For example, a pharmaceutically acceptable vehicle can be a diluent, adjuvant, excipient, or vehicle with which the therapeutic agent is administered. These vehicles can be sterile liquids, such as water and oils, including those of petroleum, animal, vegetable or synthetic origin, such as peanut oil, soybean oil, mineral oil, sesame oil, etc. Saline solutions and aqueous solutions of dextrose and glycerol can also be used as liquid vehicles, especially for injection solutions. Pharmaceutically acceptable excipients comprise starch, glucose, lactose, sucrose, sodium stearate, glycerol monostearate, talc, sodium chloride, skimmed milk powder, glycerol, propylene glycol, water, ethanol and the like. When the pharmaceutical composition is suitable for oral administration, the tablets or capsules can be prepared by conventional means with pharmaceutically acceptable excipients such as binding agents (for example pregelatinized corn starch, polyvinylpyrrolidone or hydroxypropyl methylcellulose); fillers (for example lactose, microcrystalline cellulose or calcium hydrogen phosphate); lubricants (for example, magnesium stearate, talc or silica); disintegrants (for example potato starch or sodium starch glycolate); or wetting agents (for example, sodium lauryl sulfate). The tablets can be coated by methods well known in the state of the art. Liquid preparations for oral administration may take the form of, for example, solutions, syrups or suspensions, or may be presented as a dry product to be reconstituted with water or another suitable vehicle before use. Such liquid preparations can be prepared by conventional means with pharmaceutically acceptable vehicles such as suspending agents (for example sorbitol syrup, cellulose derivatives or hydrogenated edible fats); emulsifying agents (for example, lecithin or acacia); non-aqueous vehicles (for example almond oil, oily esters, ethyl alcohol or fractionated vegetable oils); and preservatives (for example methyl or propyl p-hydroxybenzoates or sorbic acid). The pharmaceutical compositions may also contain buffering salts, flavoring agents, dyes and sweeteners, as appropriate.

The term “treating” or “treatment” encompasses any beneficial or desirable effect on a pathology or pathological condition, and may even include minimal reduction of one or more measurable markers of the pathology or pathological condition. The treatment may, for example, involve either reducing or improving the symptoms of the pathology or pathological condition, or delaying the progression of the pathology or pathological condition. The term “treatment” does not necessarily mean the complete eradication or cure of the pathology, nor of the associated symptoms.

Flatworm and Composition Containing Same

The description relates to a flatworm fed exclusively with malted yeast, said flatworm being free of human-pathogenic microorganisms.

The inventors realized that flatworms fed exclusively with malted yeast were free of human-pathogenic microorganisms, whereas flatworms fed with conventional food, such as calf liver, or even fed with baker's yeast (Saccharomyces cerevisiae), develop human-pathogenic microorganisms in their digestive tract.

The inventors have therefore shown that it suffices to feed flatworms exclusively with malted yeast to obtain flatworms free of human-pathogenic microorganisms.

Thus, the present invention does not require the use of antibiotics to obtain flatworms free of human-pathogenic microorganisms. The flatworms of the present description are therefore not only free of human-pathogenic microorganisms, but also free of antibiotics.

Flatworms can be alive or dead. It can for example be frozen, freeze-dried and/or dried.

According to the present description, the flatworm is advantageously free of the following human-pathogenic microorganisms: Aeromonas veronii, Staphylococcus capitis, Pseudomonas fluorescens, Acinetobacter guillouiae, Aeromonas hydrophila, Delftia acidovorans, Comamonas testosteroni, Sphingomonas paucimobilis, Staphylococcus epidermidis, Micrococcus luteus and Staphylococcus haemolyticus.

The invention also relates to a composition comprising one or more flatworms fed exclusively with malted yeast, said flatworm(s) being free of human-pathogenic microorganisms.

It may be, for example, a composition comprising the flatworm(s) in their environment, for example a composition comprising water, at least one flatworm according to the invention and optionally malted yeast. It may for example be a composition comprising one or more flatworms according to the present description and exclusively malted yeast as food for the flatworm(s).

The composition can be in various forms, for example in liquid form or in freeze-dried form. In freeze-dried form, the composition may additionally comprise a lyoprotectant, such as an unnatural lyoprotectant.

Method for Feeding Flatworms and Method for Preparing a Flatworm Extract

An object of the present invention relates to a method for feeding flatworms, in which the flatworms are fed exclusively with malted yeast.

The inventors have observed that it is very difficult to be able to sustainably feed flatworms with a food free of any element of animal origin. Indeed, flatworms refuse to feed with the majority of foods free of elements of animal origin.

For example, the inventors tested the following foods: baker's yeast, spirulina, soy protein, soy milk, tofu, oatmeal, malt and none of these foods was accepted as perennial food by flatworms. Consequently, these foods do not allow to obtain one or more flatworms free of element of animal origin.

In addition, these foods had no impact on the flatworm microbiota and in particular on the population of human-pathogenic bacteria.

Surprisingly, while flatworms refuse to feed on a long-term basis with malt or baker's yeast (Saccharomyces cerevisiae), the Applicant has demonstrated that flatworms accept to feed on a long-term basis with malted yeast (Saccharomyces cerevisiae with malt). Flatworms agree to feed exclusively with malted yeast, which advantageously allows to obtain flatworms not only free of human-pathogenic microorganisms but also free of element of animal origin.

The feeding method according to the invention comprises a step which consists in feeding the flatworms for a sufficient time to eliminate the human-pathogenic microorganisms and/or eliminate the elements of animal origin.

In a particular embodiment, the flatworms are fed at least once a week, preferably at least twice a week, with an amount of malted yeast ranging from 0.10 mg of yeast/400 flatworms to 0.20 mg of yeast/400 flatworms, preferably an amount of 0.15 mg yeast/400 flatworms.

Flatworms are fed for the time necessary to be free of human-pathogenic microorganisms. In a particular embodiment, the flatworms are fed for at least one week, preferably for at least 2 weeks, for example for 4 weeks.

Advantageously, the flatworms are deprived of all food for at least one week before being fed with the malted yeast.

Another object of the present invention relates to a method for preparing a flatworm extract, comprising the steps of:
(i) feeding flatworms by implementing the feeding method according to the invention, and
(ii) preparing a flatworm extract from the flatworms obtained in step (i).

Step (ii) of preparing the extract is described in more detail in the “flatworm extract” section below.

Flatworm Extract

Another object of the invention relates to a flatworm extract fed exclusively with malted yeast which can be obtained by implementing the method for preparing a flatworm extract according to the invention, said flatworms being free of human-pathogen microorganisms.

The extract according to the invention can for example be an aqueous extract.

Since flatworms are fed exclusively with malted yeast, the extract according to the invention is free of antibiotics. The absence of antibiotic within the extract according to the invention makes it particularly suitable for use in humans or animals. In addition, the extract according to the invention is free of elements of animal origin other than those relating to the flatworm itself. This advantageously allows to have an extract that is particularly suitable for use in vegan products, that is to say products not comprising any ingredient of animal origin, such as vegan cosmetic products.

The preparation of a flatworm extract does not have any particular difficulty, numerous extraction methods being described in the literature. The preparation of a flatworm extract is not limited to a particular method, and the conventional methods can be implemented to prepare an extract according to the invention, such as for example by implementing a method comprising the following steps: (i) grinding flatworms in order to obtain a ground material, (ii) ridding the ground material of insoluble solid debris in order to obtain an extract, and (iii) optionally concentrating or diluting the extract.

Step (i) can be implemented with any grinding method allowing to burst the flatworm cells and to release their contents, for example by shaking flatworms with glass beads.

Step (ii) can be implemented by centrifuging and/or filtering the ground material. Advantageously, the extract according to the invention is cleared of any insoluble solid debris, in particular cleared of membrane debris resulting from the bursting of flatworm cells. To obtain such an extract, the preparation method may comprise at least one solid/liquid separation step, for example one or more membrane filtration steps having an appropriate cut-off threshold, for example a cut-off threshold of 1.20 μm, for example 0.80 μm, 0.45 μm, 0.2 μm. Filtration allows to obtain an extract which contains particles of a size less than the cut-off threshold of the filter used, for example particles of a size of less than 1.20 μm, for example less than 0.80 μm, less than 0.45, less than μm 0.2 μm.

Step (iii) can be implemented by evaporation or by adding a suitable solvent, for example water.

Example 3 describes a method for preparing a flatworm extract according to the invention.

The extraction conditions will be chosen such that the extract obtained has the desired concentration in the intended application. The protein concentration of the crude extract can vary in particular depending on the amount of flatworm used (for example, during an aqueous extraction, when the ratio flatworm/water is increased, the protein concentration of the extract also increases), the extraction time (for example, an increase in the extraction time during an aqueous extraction generally allows to increase the protein concentration of the extract) and/or the extraction temperature (for example, an increase in the extraction temperature during an aqueous extraction generally allows to increase the protein concentration of the extract). Generally, the crude extract comprises from 1 mg to 50 mg of proteins per mL of extract (mg/mL). The absorbance at the characteristic wavelength of 280 nm (that is to say A280 nm) is commonly used to estimate the total protein concentration in the extract.

The extract obtained can be more or less concentrated in proteins depending on the intended use. After extraction, the extract may in particular comprise between 15 mg of protein per mL of extract (mg/mL) and 20 mg/mL. The crude extract can be diluted or even concentrated depending on the intended use. In a particular embodiment, the extract according to the invention comprises from 15 μg of protein per mL of extract (μg/mL) to 50 mg of protein per mL of extract (mg/mL), for example from 15 μg/mL to 20 mg/L, for example from 15 μg/mL to 1 mg/L, for example from 15 μg/mL to 0.3 mg/L. In another particular embodiment, the extract according to the invention comprises less than 0.5 mg of protein per mL of extract (mg/mL), for example less than 0.3 mg/mL, for example between 15 μg/mL and 0.3 mg/mL, preferably 15 μg mg/mL to 75 μg/mL. Complete dehydration of this extract allows to obtain an extract in powder form.

In a very particular embodiment, the extract according to the invention has a MALDI TOF spectrum corresponding to the data shown in Table 1.

TABLE 1

| Extract 1 | | | Extract 2 | | | Extract 3 | | |
|---|---|---|---|---|---|---|---|---|
| m/z | Intensity | S/N | m/z | Intensity | S/N | m/z | Intensity. | S/N |
| 2001 | 5051 | 8 | 2000.7 | 5566 | 8 | 2001.2 | 1706 | 7 |
| 2390.4 | 3699 | 5 | 2723.3 | 3988 | 7 | 2723.8 | 2062 | 9 |
| 2723.7 | 4012 | 8 | 3335.8 | 3074 | 5 | 3335.7 | 1224 | 5 |
| 3335.6 | 2812 | 5 | 3672.7 | 3390 | 7 | 3673.2 | 2021 | 9 |
| 3673.3 | 3533 | 9 | 3708.1 | 4153 | 10 | 3708.4 | 2804 | 13 |
| 3708.7 | 4596 | 13 | 4551.1 | 2822 | 7 | 3760.5 | 1500 | 7 |
| 3760.9 | 2701 | 6 | 4871.2 | 3613 | 10 | 4550.7 | 1414 | 7 |
| 4551.4 | 2604 | 7 | 5012.3 | 2930 | 8 | 4871.1 | 2254 | 11 |
| 4871.6 | 3185 | 10 | 5072.5 | 3321 | 9 | 5012.6 | 1683 | 9 |
| 5012.9 | 2746 | 8 | 5267.7 | 2351 | 6 | 5060 | 974 | 5 |
| 5060.4 | 2042 | 5 | 5646.8 | 7211 | 28 | 5072.8 | 2096 | 11 |
| 5072.8 | 3282 | 10 | 5870.1 | 2197 | 6 | 5268 | 1410 | 7 |
| 5268.3 | 2221 | 6 | 5890.7 | 2105 | 6 | 5647.1 | 5163 | 30 |
| 5647.6 | 6601 | 28 | 5906.5 | 2005 | 6 | 5870.4 | 1070 | 6 |
| 5871.3 | 1808 | 5 | 6393.6 | 5383 | 23 | 5890.8 | 1067 | 6 |
| 5891.4 | 1859 | 6 | 6415.4 | 1900 | 6 | 5906.7 | 1008 | 6 |
| 5907.7 | 1886 | 6 | 6432.8 | 4926 | 20 | 5965 | 901 | 5 |
| 5965.5 | 2019 | 6 | 6487 | 2204 | 7 | 6393.6 | 3673 | 24 |
| 6394.2 | 4603 | 21 | 6534.1 | 3192 | 12 | 6414.6 | 882 | 6 |
| 6433.5 | 4207 | 19 | 6556.8 | 4018 | 16 | 6433.2 | 3106 | 20 |
| 6487.8 | 2003 | 7 | 6577.9 | 1864 | 6 | 6487.1 | 1245 | 8 |
| 6535 | 2888 | 12 | 6674.1 | 5905 | 27 | 6534.4 | 1980 | 13 |
| 6557.6 | 3510 | 15 | 6695.2 | 1817 | 6 | 6557 | 2491 | 16 |
| 6675.2 | 4693 | 23 | 7347.6 | 7866 | 44 | 6674.5 | 3967 | 28 |
| 7306.6 | 1498 | 5 | 7369.5 | 2976 | 14 | 7348 | 6232 | 50 |
| 7349 | 7585 | 45 | 7418 | 10220 | 58 | 7369.7 | 2025 | 16 |
| 7369.9 | 2881 | 15 | 7438.6 | 3214 | 15 | 7418.4 | 7822 | 62 |
| 7419.5 | 8974 | 54 | 7471.5 | 2704 | 12 | 7472 | 1966 | 16 |
| 7472.7 | 2731 | 14 | 7492.2 | 2184 | 9 | 7523 | 4434 | 37 |
| 7524.1 | 5714 | 35 | 7522.9 | 5581 | 31 | 7542.1 | 1296 | 11 |
| 7568 | 1726 | 7 | 7541.6 | 2177 | 10 | 7569.3 | 1048 | 9 |
| 7588.1 | 3819 | 22 | 7564.9 | 1721 | 7 | 7587.1 | 2747 | 23 |
| 7606.4 | 1577 | 6 | 7586.4 | 3623 | 19 | 7754.2 | 616 | 5 |
| 7755.1 | 1452 | 6 | 7607 | 1645 | 6 | 8470.5 | 1655 | 17 |
| 8471.3 | 2189 | 15 | 8469.5 | 2390 | 16 | 8498.9 | 806 | 8 |
| 8499.6 | 1422 | 8 | 8498.3 | 1731 | 11 | 8525.6 | 870 | 9 |
| 8526.7 | 1394 | 8 | 8523.9 | 1798 | 11 | 8545.9 | 642 | 7 |
| 8547 | 1154 | 6 | 8544.4 | 1268 | 7 | 9395.7 | 741 | 9 |
| 9396.7 | 1148 | 9 | 9395.2 | 1143 | 8 | 14027.7 | 146 | 6 |
| 14031.1 | 223 | 5 | | | | | | |

M/z: ratios (mass in Dalton/charge) on the abscissa of the different characteristic peaks of the spectrum; S/N: ratios (peak intensity/background noise intensity) of the different characteristic peaks of the spectrum.

Composition Comprising a Flatworm Extract

The invention also relates to a composition comprising an extract according to the invention.

In a first particular embodiment, the composition is a cosmetic and/or dermatological composition and may comprise one or more carriers and/or excipients suitable for the targeted use. In this embodiment, the composition can be in the form of a gel, lotion, cream, ointment, paste or soap. Thus, the composition according to the invention may comprise various usual excipients suitable for external topical administration, in particular excipients that are dermatologically and/or cosmetologically acceptable. These excipients suitable for the formulation are well known to the person skilled in the art and comprise, for example, agents promoting penetration at the skin, moisturizing agents, thickening agents, stabilizing agents, emollient agents and surfactants, emulsifying agents, preservatives, pH adjusting agents, etc. For example, the composition according to the invention is formulated in the form of a cream comprising the extract of the invention and at least the following excipients: an emulsifying agent, an emollient agent, a thickening agent, a moisturizing agent and optionally a preservative. In another example, the composition according to the invention is formulated in the form of a cream comprising the extract of the invention and at least the following additional excipients: a gelling agent, a thickening agent, a moisturizing agent and optionally a preservative. In this first embodiment, the composition according to the invention may also comprise other compounds suitable for cosmetic and/or dermatological use, such as radical scavengers, antioxidant vitamins such as vitamin E, vitamin C, antioxidant agents such as natural polyphenols, enzymes, plant active ingredients, natural anti-inflammatory substances, alcohols, polyols, esters, electrolytes, polar and non-polar oils, polymers, copolymers, phospholipids, dyes, perfumes and/or skin peeling agents.

In a second particular embodiment, the composition is a nutraceutical composition (or food supplement) and may comprise one or more carriers and/or excipients suitable for the targeted use. In this embodiment, the composition must therefore be able to be ingested. It can be in different dosage forms, such as a dispersion, a capsule, a cachet, a powder, a tablet, etc. The nutraceutical composition can also be in the form of a food, such as a yoghurt, a cereal bar, a gel, a candy, a chewing gum, etc.

In a third particular embodiment, the composition is a pharmaceutical composition and may comprise a pharmaceutically acceptable vehicle. Pharmaceutically acceptable excipients comprise starch, glucose, lactose, sucrose, sodium stearate, glycerol monostearate, talc, sodium chloride, dried skim milk, glycerol, propylene glycol, water, ethanol and the like. When the pharmaceutical composition is suitable for oral administration, the tablets or capsules may be prepared by conventional means with pharmaceutically acceptable excipients such as binding agents (for example pregelatinized corn starch, polyvinylpyrrolidone or hydroxypropyl methylcellulose); bulking agents (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulfate). Tablets can be coated by methods well known in the art. Liquid preparations for oral administration may take, for example, the form of solutions, syrups or suspensions, or they may be presented as a dry product to be reconstituted with water or another suitable vehicle before use. These liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffering salts, flavoring agents, dyes and sweeteners, as appropriate.

The composition according to the invention may also comprise a cell culture medium, preferably a culture medium capable of cultivating human or animal cells.

Method for Eliminating Pathogenic Microorganisms Present in a Flatworm

The invention also relates to a method for eliminating pathogenic microorganisms present in one or more flatworms, said method comprising a step which consists in feeding one or more flatworms, preferably exclusively, with malted yeast for a time sufficient to eliminate said pathogenic microorganisms. Advantageously, the method according to the invention does not require the use of antibiotics in order to have a flatworm free of human-pathogenic microorganisms.

The method allows in particular to obtain one or more flatworms according to the invention, as defined in the section “Flatworm and composition containing same” above.

In a particular embodiment, the flatworms are fed at least once a week, preferably at least twice a week, with an amount of malted yeast ranging from 0.10 mg of yeast/400 flatworms to 0.20 mg of yeast/400 flatworms, preferably an amount of 0.15 mg yeast/400 flatworms.

Flatworms are fed for the time necessary to be free of human-pathogenic microorganisms. In a particular embodiment, the flatworms are fed for at least one week, preferably for at least 2 weeks, for example for 4 weeks.

Advantageously, the flatworms are deprived of all food for at least one week before being fed with the malted yeast.

Method for Preparing a Flatworm Extract

The invention also relates to a method for preparing a flatworm extract according to the invention, comprising the steps of:
(a) obtaining flatworms free of human-pathogenic microorganisms by implementing the method for eliminating pathogenic microorganisms present in one or more flatworms according to the invention;
(b) preparing a flatworm extract from the flatworms obtained in step (a).

Step (b) preparing the extract is described in more detail in the section “Flatworm extract” above.

Non-Therapeutic Indications

The invention also relates to the use of an extract according to the invention or of a composition comprising an extract according to the invention, to cultivate human or animal cells in vitro or ex vivo. In particular, the invention relates to the use of an extract according to the invention or of a composition comprising an extract according to the invention, for cultivating human or animal cells in vitro or ex vivo, in which said human cells are not human embryonic stem cells requiring the destruction of a human embryo and having the capacity to develop into a human being.

The extract can for example be used to regenerate human or animal cells, for example by stimulating their differentiation, their growth, their multiplication and/or their proliferation. According to a particular aspect, the extract is used to regenerate human dental stem cells.

The human or animal cells can be any type of cell, for example stem cells or differentiated cells. When the stem cells considered are human stem cells, the latter are not human embryonic stem cells requiring the destruction of a human embryo and having the capacity to develop into a human being.

Among the differentiated cells, reference may in particular be made to epithelial cells, fibroblasts, keratinocytes, endothelial cells, neuronal cells or nerve cells.

Another object of the present invention relates to the use of an extract according to the invention or of a composition comprising an extract according to the invention as an anti-aging agent, healing agent and/or agent for regenerating human or animal cells, wherein said extract or said composition is applied topically. The term “topical route” refers to an application to the skin, the mucous membranes, the superficial body growths such as the nails, body hair and hair, preferably the skin, for example an application to healthy skin.

The person skilled in the art will be able to determine the amount and frequency of application depending on the anti-ageing, healing and/or regenerating effect sought and the type of cells targeted. The anti-ageing, healing and/or regenerating effects can be measured by analyzing the expression of one or more genes whose role in cell ageing, healing or else cell proliferation is known.

It is also possible to describe here a cosmetic method for treating the skin, for example healthy skin, to combat the signs of skin ageing, and in particular the expression wrinkles caused by uncontrolled facial muscle contractions, consisting in applying to the areas of the skin requiring such treatment, a cosmetic composition according to the invention.

Therapeutic Indications

The invention also relates to an extract according to the invention or a composition comprising an extract according to the invention, for the use thereof as a medicament.

The extract or composition can be formulated for parenteral administration, for example intravascular (intravenous or intra-arterial), intraperitoneal, intramuscular, enteral or topical administration.

The administration or the application can be done at once or, more generally, in several times. The duration of treatment may vary depending on the pathology being treated and the subject being treated.

The extract or the composition can be used in monotherapy or in combination with drugs whose therapeutic interest is recognized in the considered pathology.

In particular, the invention relates to an extract according to the invention or a composition comprising an extract according to the invention, for the use thereof to promote the healing of a skin wound or a skin burn. Topical administration is preferred in this indication.

### EXAMPLES

### Example 1: Analysis of the Microorganisms Contained in Planarians Fed with Different Diets

a) Planarians Belonging to the Schmidtea mediterranea Species Fed with Calf Liver or Malted Yeast

A first group of 400 planarians (Schmidtea mediterranea) was fed twice a week, for 2 weeks, with 0.15 g of malted yeast (Markal—Malt beer yeast—Flakes 250 g), that is to say 0.0375 g/100 planarians/feeding.

A second group of 400 planarians (Schmidtea mediterranea) was fed twice a week, for 2 weeks, with calf liver (SAPRIMEX company, Italy) at 0.40 mg/400 planarian/feeding.

The microorganisms contained in the two groups of planarians were analyzed and identified by analyzing the microbiota according to the protocol detailed below.

Microbiota Analysis Protocol

After two weeks of starvation, the planarians were washed in water sterilized by filtration, then the planarians were inoculated on BCYE agar (Oxoid Deutschland GmbH, Wesel, Germany), Luria Bertani (LB) or Columbia agar enriched with 5% sheep blood (bioMérieux, Marcy l'étoile, France). Each of the inoculations is incubated under three different temperature conditions (19° C., 28° C. and 37° C.) and under two conditions of incubation time (24 h and 48 h), namely 24 h at 19° C., 24 h at 28° C., 24 h at 37° C., 48 h at 19° C., 48 h at 28° C. or 48 h at 37° C. Each individual bacterial colony was harvested and identified by MALDI-TOF-MS (Microflex Spectrometer; Bruker Daltonics, Bremen, Germany). The spectra obtained were imported into the MALDI Biotyper 3.0 software (Bruker Daltonics) and analyzed with respect to the reference spectra of the bacteria included in the database (Bruker database constantly updated with the Mediterranee-Infection database. The MALDI Biotyper RTC software was used to interpret the results according to scores obtained: a colony was probably identified at the species for a score >2.0, probably identified for a score between 1.99 and 1.7, but not identified for a score <1.7.

Bacterial colonies that were identified using MALDI-TOF, but which were not known in the Bruker database, were identified by phylogenetic analysis, using 16S rRNA sequencing. Genomic DNA was extracted using an EZ1 automaton and the DNA tissue kit (Qiagen, Hilden, Germany). Full amplification and sequencing of the 16S rRNA gene was performed using eight primers on an ABI Prism 3130xl Genetic Analyzer capillary sequencer (Applied Bio systems, Bedford, MA, USA). CodonCode Aligner software was used for sequence alignment, assembly and correction (https://www.codoncode.com/). For taxonomic assignment, a BLASTn search was performed on database nr. A sequence similarity threshold of 98.65% compared to the phylogenetically closest species with the position in the nomenclature was used to delineate a new species. Phylogenetic relationships were deduced from 16S rRNA sequence comparison using MEGA7 software.

Results of the Microbiota Analysis

Planarian fed with malted yeast were free of bacteria likely to be pathogenic for humans. Planarians fed with malted yeast comprised a single bacterial species, non-pathogenic to humans: Pseudomonas brenneri.

It is interesting to note that (non-pathogenic) bacteria Urmitella massiliensis, Paenibacillus polymyxa and Micrococcus luteus were found in malted yeast fed to planarians, but these bacteria were not found in planarians after feeding with malted yeast.

Planarians fed with calf liver contained 11 bacteria likely to be pathogenic for humans (Table 2) and 25 non-pathogenic bacteria (Table 3).

TABLE 2
Pathogenic bacteria found in planarians fed with calf liver

Aeromonas veroni
Staphylococcus capitis
Pseudomonas fluorescens
Acinetobacter guillouiae
Aeromonas hydrophila
Delftia acidovorans
Comamonas testosteroni
Sphingomonas paucimobilis
Staphylococcus epidermidis
Micrococcus luteus
Staphylococcus haemolyticus

TABLE 3
Non-pathogenic bacteria found in planarians fed with calf liver

Pedobacter ghigonii Marseille-Q2390 (*)
Pseudomonas schmidteae Marseille-Q1929 (*)
Pseudomonas timonensis Marseille-P9894
Pseudomonas timonensis Marseille-P9899
Chryseobacterium schmidteae Marseille-P9602 (*)
Bacillus schmidteae Marseille-P9898
Janthinobacterium timonensis Marseille-P9896
Vogesella uthrealis
Pedobacter schmidteae EG (*)
Comamonas aquatilis (*)
Pedobacter wanjuense
Pseudomonas brenneri
Variovorax paradoxus
Pseudomonas anguilliseptica
Chryseobacterium balustine
Chryseobacterium scophthalmum
Agrobacterium tumefaciens
Microbacterium oxydans
Micrococcus yunnanensis
flavobacterium oncorhynchi
flavobacterium tructae
Pseudomonas gessardii
shinella zoogloeoides
Acinetobacter bereziniae
ensifer adhaerens
Rhizobium giardinii
Corynebacterium lipophiloflavum
Acidovorax wautersii
Sphingomonas bisphenolicum
Sphingomonas ginsenosidimutans
(*) Newly identified bacteria

It should be noted that the non-pathogenic bacteria Brochothrix thermosphacta, Lactococcus piscium, Pseudomonas frederiksbergensis, Pseudomonas gessardii, Serratia proteamaculans, Staphylococcus hominis, and Pseudomonas azotoformans have been found in calf liver given to planarians, but these bacteria were not found in planarians after feeding with calf liver.

Conclusion: feeding the planarians with malted yeast allowed to eliminate the microorganisms likely to be pathogenic for humans, and therefore to obtain planarians free of human-pathogenic microorganisms. These planarians free of human-pathogenic microorganisms are particularly suitable for the preparation of a planarian extract which does not have a risk for humans.

b) Planarians Belonging to the Dugesia japonica Species Fed with Calf Liver or Malted Yeast

1) Material and Method

The material and method of this test is identical to that implemented in example 1 of the patent application with planarians belonging to the Schmidtea mediterranea species but with planarians belonging to the Dugesia japonica species.

2) Results of the Microbiota Analysis

Planarians fed with calf liver contained 9 bacteria pathogenic for humans (Table 4 reproduced below).

TABLE 4
Human-pathogenic bacteria found in planarians fed with calf liver

Aeromonas veroni
Staphylococcus capitis
Pseudomonas fluorescens
Acinetobacter guillouiae
Aeromonas hydrophila
Delftia acidovorans
Comamonas testosteroni
Staphylococcus epidermidis
Staphylococcus haemolyticus

Feeding Dugesia japonica exclusively with malted yeast allowed the elimination of all human-pathogenic bacteria detected when the planarians are fed with calf liver. No pathogenic bacteria for humans have been detected in Dugesia japonica fed exclusively with malted yeast.

3) Conclusion

Feeding Dugesia japonica planarians exclusively with malted yeast has eliminated human-pathogenic microorganisms. These planarians free of human-pathogenic microorganisms are particularly suitable for the preparation of a planarian extract which does not have a risk for humans.

c) Planarians Belonging to the Schmidtea mediterranea Species Fed with Heat-Inactivated Malted Yeast

1) Material and Method

The material and method of this test is the same as that implemented in example 1 of the patent application but by feeding the planarians exclusively with inactivated malted yeast and not exclusively with live malted yeast.

The malted yeast is inactivated by heat treatment at a temperature of 50° C. for 1 hour.

2) Results of the Microbiota Analysis

Planarians belonging to the Schmidtea mediterranea species fed exclusively with inactivated malted yeast contain bacteria that are pathogenic for humans, namely:

TABLE 5
Human-pathogenic bacteria found in planarians
fed with inactivated malted yeast

Aeromonas veroni
Staphylococcus capitis
Pseudomonas fluorescens
Acinetobacter guillouiae
Aeromonas hydrophila
Delftia acidovorans
Comamonas testosteroni
Sphingomonas paucimobilis
Staphylococcus epidermidis
Micrococcus luteus
Staphylococcus haemolyticus

3) Conclusion

Feeding planarians with inactivated malted yeast does not eliminate the human-pathogenic microorganisms present in Schmidtea mediterranea.

d) Planarians Belonging to the Schmidtea mediterranea Species Fed with Baker's Yeast (Saccharomyces cerevisiae)

1) Material and Method

The material and method of this test is the same as that implemented in example 1 of the patent application but by feeding the planarians exclusively with baker's yeast Saccharomyces cerevisiae and not exclusively with malted yeast (Saccharomyces cerevisiae+malt according to the invention).

2) Results of the Microbiota Analysis

Planarians belonging to the Schmidtea mediterranea species fed exclusively with baker's yeast contain bacteria that are pathogenic for humans, namely:

TABLE 6
Human-pathogenic bacteria found in
planarians fed with baker's yeast

Aeromonas veroni
Staphylococcus capitis
Pseudomonas fluorescens
Acinetobacter guillouiae
Aeromonas hydrophila
Delftia acidovorans
Comamonas testosteroni
Sphingomonas paucimobilis
Staphylococcus epidermidis
Micrococcus luteus
Staphylococcus haemolyticus

3) Conclusion

Feeding planarians with baker's yeast does not eliminate the human-pathogenic microorganisms present in Schmidtea mediterranea.

### Example 2: Tests of Different Diets without Element of Animal Origin on Planarians Belonging to the Schmidtea mediterranea Species

Planarians belonging to the Schmidtea mediterranea species are fed according to the same method as in Example 1 part a) with different foods without element of animal origin. The different foods tested are as follows: baker's yeast, spirulina, tofu, soy protein, soy milk, oatmeal or malt.

The results obtained are reproduced in the table below.

TABLE 7

Amount (mg/400 planarian)

| | | |
|---|---|---|
| 0.10 | 0.15 | 0.20 |

| | |
|---|---|
| Type of food | Palatability |

| | | | |
|---|---|---|---|
| Baker's yeast | − | +/− (*) | − |
| Malted yeast | + | + | + |
| Malt | − | − | − |
| Spirulina | − | − | − |
| [symbol] oy protein | − | − | − |
| Soy milk e | − | − | − |
| Tofu | − | − | − |
| [symbol] atmeal | − | − | − |

* after (*) after two feedings planarians refuse to feed on baker's yeast

Platyhelminthes do not feed in a perennial way with any of these foods without element of animal origin. Surprisingly, flatworms do not accept feeding with malt unlike malted yeast. Feeding flatworms with each of the foods free of elements of animal origin, except malted yeast, does not eliminate the human-pathogenic microorganisms present in Schmidtea mediterranea.

Surprisingly still, flatworms do not accept perennial feeding with baker's yeast (Saccharomyces cerevisiae) but accept feeding with malted yeast (Saccharomyces cerevisiae with malt), which differs from baker's yeast only by the presence of malt.

### Example 3: Preparation of the Planarian Extract Fed with Malted Yeast

1. Preparation of Planarians

About 400 flatworms (Schmidtea mediterranea) fed with malted yeast according to Example 1 (wet mass comprised between 0.1 g and 0.4 g) were starved for 2 weeks.

The planarians were washed twice with 200 ml of sterile water (ceramic: microfiltration (0.2 micron), interior of the antibacterial ceramic, compacted activated carbon). The washed planarians were transferred to a tube and the water was removed then the planarians were frozen at −80° C.

2. Obtaining the Extract

The tube containing the frozen planarians was placed on ice until thawed. 20 ml of sterile water (temperature 4° C.) was added to the thawed planarians. 1 cm of sterile glass ball (diameter 3 mm) was added to the tube (tube A).

Tube A was mixed using a mechanical stirrer for 5 minutes at 2500 cycles/minute. The content of tube A was passed through a syringe equipped with a 23 g needle and placed in a new tube (tube B).

20 ml of sterile water (temperature of 4° C.) was added to tube A containing the glass beads and the residues which remained stuck to the beads. Tube A was again mixed using a mechanical stirrer for 5 minutes at 2500 cycles/minute. The content of tube A was again passed through a syringe equipped with a 23 g needle then transferred to tube B. Tube B now contains 40 ml of crude planarian extract.

Tube B is centrifuged for 5 min at 3220 g at 4° C. then the supernatant of tube B was filtered through a 1.20 μm membrane (filtrate 1). Filtrate 1 was then filtered on a 0.80 μm membrane (filtrate 2). Filtrate 2 was then filtered on a 0.45 μm membrane (filtrate 3). Filtrate 3 was then filtered on a 0.2 μm membrane (filtrate 4). Filtrate 4 only contained particles smaller than 0.2 μm.

The protein concentration of filtrate 4 was determined by absorbance at the wavelength of 280 nm. For 400 initial planarians (wet mass comprised between 0.1 g and 0.4 g) the protein concentration obtained was comprised between 17.5 and 20 mg of protein/mL.

The filtrate 4 (corresponding to an aqueous extract freed from any insoluble solid debris larger than 0.2 μm) was freeze-dried and stored at 4° C. for subsequent use.

Filtrate 4 was analyzed in triplicate by MALDI-TOF according to the protocol detailed below.

MALDI-TOF Analysis Protocol

A 1 μl sample of aqueous extract from example 4A was diluted to 1/20th with a mixture of trichloroacetic acid, acetonitrile and water and was deposited on an HCCA matrix. Then, 1 μL of HCCA saturated matrix was added on top, then the whole was dried for 10 minutes. The analysis of the extract was performed with a Maldi-Tof Microflex LT10 (Bruker) mass spectrometer. The results were collected with the MALDl Biotype RTC software.

The spectra acquisition method comprised the following steps and characteristics:
introduce the plate (Microflex target reference model 224989 called M5P 96) containing the depositions into the Microflex LT mass spectrometer,
start the computer associated with the spectrometer to launch the analyzes and
launch the acquisition of MALDl-TOF mass spectra.

The parameters applied to the mass spectrometer were as follows: positive linear model Electrode 151: 20.00 kV, Electrode 152: 18.05 kV, Focusing electrode: 6 kV1 Laser frequency: 60 Hz, Detector gain: 8.8×, Post Ion Extraction (PIE): 120 ns, Mass range: from 700 to 20000 Da.

The spectra were obtained using an automatic method controlled by the FLEXCONTROL® software from the manufacturer BRUKER DALTONICS® the parameters were as follows for each spot: number of series of 40 shots: 6 on different positions of the spot (6×40=240 spectra), for the shots to sweep the entire sample in a homogeneous way the acquisition is done on 6 different positions according to a hexagonal geometry, pre-shots: 10 shots at 40% of the maximum laser power are used to desalinate the sample (contamination by mineral salts), laser power: 30% to 45% regulated via the FLEXCONTROL® software, spectrum selection: over a mass range of 2000 to 20000 Da. The acquisition of the spectra is ultimately carried out using the BIOTYPER® software from the manufacturer BRUKER DALTONICS® which allows to apply the automatic method described above to a series of samples taking into account the criteria mentioned above and a quality score or validation score which represents the addition of 2 ratings nl+n2 (n1 being a function of the number of peaks and n2 a function of the signal/background noise ratio):
nl is the rating assigned according to the number of characteristic peaks of the spectrum n, with: nl=0 for n::s: 8, nl=1 for 8<n::s: 27, nl=2 for 27<n:s; 43, nl=3 for 43<n:s; 78, nl=4 for 78<n:;; 86, nl=5 for 86<n:;; 110, nl=6 for 110<n, and
n2 is the rating assigned according to the value of the signal-to-noise ratio (Rmax) of the peak of greatest intensity in the spectrum, with n2=0 for Rmax:s; 6.8, n2=1 for 6.8<Rmax::s: 26.8, n2=2 for 26.8<Rmax::s: 52.6, n2=3 for 52.6<Rmax::s: 208.6, n2=4 for 208.2<Rmax:::; 398.6, n2=5 for 398.6<Rmax::s: 1092.2, n2=6 for 1092.2<Rmax.

A spectrum is taken into account if its validation score (nl+n2) is greater than 2.

Results of MALDI-TOF Analysis

The data are shown in Table 8 (m/z: mass to charge, intensity: signal intensity; S/N: signal to noise).

TABLE 8

| Experiment 1 | | | Experiment 2 | | | Experiment 3 | | |
|---|---|---|---|---|---|---|---|---|
| m/z | Intensity | S/N | m/z | Intensity | S/N | m/z | Intensity. | S/N |
| 2001 | 5051 | 8 | 2000.7 | 5566 | 8 | 2001.2 | 1706 | 7 |
| 2390.4 | 3699 | 5 | 2723.3 | 3988 | 7 | 2723.8 | 2062 | 9 |
| 2723.7 | 4012 | 8 | 3335.8 | 3074 | 5 | 3335.7 | 1224 | 5 |
| 3335.6 | 2812 | 5 | 3672.7 | 3390 | 7 | 3673.2 | 2021 | 9 |
| 3673.3 | 3533 | 9 | 3708.1 | 4153 | 10 | 3708.4 | 2804 | 13 |
| 3708.7 | 4596 | 13 | 4551.1 | 2822 | 7 | 3760.5 | 1500 | 7 |
| 3760.9 | 2701 | 6 | 4871.2 | 3613 | 10 | 4550.7 | 1414 | 7 |
| 4551.4 | 2604 | 7 | 5012.3 | 2930 | 8 | 4871.1 | 2254 | 11 |
| 4871.6 | 3185 | 10 | 5072.5 | 3321 | 9 | 5012.6 | 1683 | 9 |
| 5012.9 | 2746 | 8 | 5267.7 | 2351 | 6 | 5060 | 974 | 5 |
| 5060.4 | 2042 | 5 | 5646.8 | 7211 | 28 | 5072.8 | 2096 | 11 |
| 5072.8 | 3282 | 10 | 5870.1 | 2197 | 6 | 5268 | 1410 | 7 |
| 5268.3 | 2221 | 6 | 5890.7 | 2105 | 6 | 5647.1 | 5163 | 30 |
| 5647.6 | 6601 | 28 | 5906.5 | 2005 | 6 | 5870.4 | 1070 | 6 |
| 5871.3 | 1808 | 5 | 6393.6 | 5383 | 23 | 5890.8 | 1067 | 6 |
| 5891.4 | 1859 | 6 | 6415.4 | 1900 | 6 | 5906.7 | 1008 | 6 |
| 5907.7 | 1886 | 6 | 6432.8 | 4926 | 20 | 5965 | 901 | 5 |
| 5965.5 | 2019 | 6 | 6487 | 2204 | 7 | 6393.6 | 3673 | 24 |
| 6394.2 | 4603 | 21 | 6534.1 | 3192 | 12 | 6414.6 | 882 | 6 |
| 6433.5 | 4207 | 19 | 6556.8 | 4018 | 16 | 6433.2 | 3106 | 20 |
| 6487.8 | 2003 | 7 | 6577.9 | 1864 | 6 | 6487.1 | 1245 | 8 |
| 6535 | 2888 | 12 | 6674.1 | 5905 | 27 | 6534.4 | 1980 | 13 |
| 6557.6 | 3510 | 15 | 6695.2 | 1817 | 6 | 6557 | 2491 | 16 |
| 6675.2 | 4693 | 23 | 7347.6 | 7866 | 44 | 6674.5 | 3967 | 28 |
| 7306.6 | 1498 | 5 | 7369.5 | 2976 | 14 | 7348 | 6232 | 50 |
| 7349 | 7585 | 45 | 7418 | 10220 | 58 | 7369.7 | 2025 | 16 |
| 7369.9 | 2881 | 15 | 7438.6 | 3214 | 15 | 7418.4 | 7822 | 62 |
| 7419.5 | 8974 | 54 | 7471.5 | 2704 | 12 | 7472 | 1966 | 16 |
| 7472.7 | 2731 | 14 | 7492.2 | 2184 | 9 | 7523 | 4434 | 37 |
| 7524.1 | 5714 | 35 | 7522.9 | 5581 | 31 | 7542.1 | 1296 | 11 |
| 7568 | 1726 | 7 | 7541.6 | 2177 | 10 | 7569.3 | 1048 | 9 |
| 7588.1 | 3819 | 22 | 7564.9 | 1721 | 7 | 7587.1 | 2747 | 23 |
| 7606.4 | 1577 | 6 | 7586.4 | 3623 | 19 | 7754.2 | 616 | 5 |
| 7755.1 | 1452 | 6 | 7607 | 1645 | 6 | 8470.5 | 1655 | 17 |
| 8471.3 | 2189 | 15 | 8469.5 | 2390 | 16 | 8498.9 | 806 | 8 |
| 8499.6 | 1422 | 8 | 8498.3 | 1731 | 11 | 8525.6 | 870 | 9 |
| 8526.7 | 1394 | 8 | 8523.9 | 1798 | 11 | 8545.9 | 642 | 7 |
| 8547 | 1154 | 6 | 8544.4 | 1268 | 7 | 9395.7 | 741 | 9 |
| 9396.7 | 1148 | 9 | 9395.2 | 1143 | 8 | 14027.7 | 146 | 6 |
| 14031.1 | 223 | 5 | | | | | | |

### Example 4: Effect of the Planarian Aqueous Extract (Filtrate 4 Obtained in Example 3)

1. Toxicity of the Planarian Aqueous Extract

The cytotoxicity of the aqueous extract obtained in Example 3 (filtrate 4) was tested on HL60 human lymphocyte cells (ATCC CCL-240). The cells were contacted with different dilutions of the extract. Briefly, 5×10⁴ cells/well (24-well plate) were cultured in RPMI medium (GibCoBRL) supplemented with fetal calf serum (10%) and penicillin and streptomycin (5000 U/ml, 5000 μg/ml respectively), at 37° C., and under a 5% CO2 atmosphere.

At a dilution of 1:5 (that is to say 0.3 mg of protein/ml of extract) the extract caused cell death. The 1:20 (that is to say 75 μg of protein/ml of extract) and 1:100 (that is to say 15 μg of protein/ml of extract) dilutions did not affect cell viability (FIG. 1).

2. Effect of Planarian Aqueous Extract on Human Stem Cell Multiplication

The cells used were human stem cells (ATCC CLS-300702). The cells were contacted with different dilutions of the extract. Briefly, 5×10³ cells/well, on a 48-well plate, were cultured in DMEM/F12 medium (GibCoBRL) supplemented with fetal calf serum (10%) and penicillin and streptomycin (5000 U/ml, 5000 μg/ml respectively), at 37° C., and under 5% CO2 atmosphere. The results are shown in FIG. 2A to D. Only a dilution (1/20; FIG. 2B), which is comprised between 1:5 (FIG. 2A) and 1:100 (FIG. 2C), allowed a significant induction (*p<0.05) of human stem cell proliferation compared to untreated cells. The induction of multiplication was about 1.6 times. FIG. 2D shows the growth of human stem cells treated with the planarian aqueous extract diluted to 1/20^{th} for 9 days and the control without treatment. The cells were stained with Giemsa.

3. Measurement of the Effect of the Planarian Aqueous Extract on the Multiplication of Human Epithelial Cells

The cells used were human epithelial cells (ATCC CCL-2). The cells were contacted with different dilutions of the extract. Briefly, 5×10³ cells/well, on a 48-well plate, were cultured in DMEM medium (GibCoBRL) supplemented with fetal calf serum (10%) and penicillin and streptomycin (5000 U/ml, 5000 μg/ml respectively), at 37° C., and under a 5% CO2 atmosphere.

A dilution (1/20; FIG. 3B), ranging from 1:5 (FIG. 3A) to 1:100 (FIG. 3C), allowed a significant induction (*p<0.05) of the proliferation of human epithelial cells compared to untreated cells. The induction of multiplication was about 1.8 times. FIG. 3D shows the growth of human epithelial cells treated with the aqueous extract diluted to 1/20^{th} for 9 days and the control without treatment. The cells were stained with Giemsa.

4. Effect of Planarian Aqueous Extract on Human Endothelial Cell Multiplication

The cells used were human endothelial cells (ATCC CRL-1730). The cells were contacted with different dilutions of the extract. Briefly, 5×10³ cells/well, on a 48-well plate, were cultured in DMEM medium (GibCoBRL) supplemented with fetal calf serum (10%) and penicillin and streptomycin (5000 U/ml, 5000 μg/ml respectively), at 37° C., and under a 5% CO2 atmosphere.

A dilution (1/20; FIG. 4B), ranging from 1:5 (FIG. 4A) to 1:100 (FIG. 4C), allowed a significant induction (*p<0.05) of the proliferation of human endothelial cells compared to untreated cells. The induction of multiplication was about 1.38 times.

5. Effect of the Aqueous Extract of Heat-Treated Planarian on the Replication of Human Stem Cells (ATCC CLS-300702), Human Endothelial Cells (ATCC CRL-1730), Human Epithelial Cells (ATCC CCL-2)

A 1/20 dilution of the planarian aqueous extract obtained in Example 3 (filtrate 4) treated at 95° C. for 10 minutes is added to the human cells of interest. Cell multiplication was measured after 9 days.

Heat inactivated the activity of the extract since no cell proliferation was observed, either for human stem cells (FIG. 5A), human epithelial cells (FIG. 5B), or human endothelial cells (FIG. 5C). The heat-inactivated planarian aqueous extract does not induce cell multiplication.

This suggests that the properties of the extract come from the proteins contained in the extract.

6. Effect of Planarian Aqueous Extract on Wound Healing of Human Stem Cell (ATCC CLS-300702) and Human Epithelial Cell (ATCC CCL-2) Cellular Layer

Cells were grown to confluence until a cellular layer was formed. A 325 μm wide lesion of the cellular layer was made using a calibrated needle, then a 1/20 dilution of the planarian aqueous extract was added or not to the cell culture. Healing of the cellular layer (complete filling of the lesion with new cells) was monitored over time.

The results are illustrated in the figures [FIG. 6A] and [FIG. 6B].

It was observed that for human stem cells, as for epithelial cells, the healing of the lesion of the cellular layer was almost complete after 24 hours, and the lesion was completely closed after 48 hours of treatment with the planarian aqueous extract diluted to 1:20^{th}. In the absence of extract, healing was not complete after 48 hours.

7. Effect of Planarian Aqueous Extract on Gene Induction in Human Cells

Human epithelial, endothelial (ATCC CRL-1730) and stem cells (ATCC CLS-300702) were treated with a 1/20^{th} dilution of the planarian aqueous extract, then the expression of certain genes was measured by quantitative PCR. The expression was expressed in “fold change”. The higher the fold change, the more the gene is expressed.

The results are shown in Table 9.

TABLE 9

| | | Ratio of gene expression relative to control | | |
|---|---|---|---|---|
| Genes | Functions | Epithelial cells | Endothelial cells | Stem cells |
| CD44 | hyaluronic acid | 1.94 | 1.47 | 2.36 |
| | receptor/immunity/adhesion and | | | |
| | cell migration | | | |
| TLR2 | Immunity | 3.21 | 2.95 | 6.04 |
| TGFb | cell proliferation | 2.64 | 1.08 | 1.73 |
| RunX1 | multiplication, tissue cohesion, cell | 2.7 | 0.4 | 9.38 |
| | differentiation | | | |
| PiwiL1 | cell renewal | 2.35 | 0.19 | 9.29 |
| SGK1 | UV protection and anti-oxidative | 29 | 4.49 | 10.92 |
| | stress, photo-aging | | | |
| FOXO1 | UV protection, photo-aging | 2.25 | 0.13 | 8.43 |
| BCL2 | anti cell death | 2.05 | 0.19 | 27.37 |
| HMMR | hyaluronic acid receptor, | 1.5 | 1.59 | 2.56 |
| | associated cell mobility | | | |
| COL1A1 | Collagen synthesis | 3.14 | 0.3 | 2.07 |
| FBL5 | Healing | 0 | 171.87 | 28.4 |

Depending on the cell type, it was observed that the planarian aqueous extract induced healing genes, immune genes, cell renewal genes, cell migration genes, genes for protection against photo-aging, anti cell death genes, and hyaluronic acid receptor activating genes.

### BRIEF DESCRIPTION OF THE DRAWINGS

FIG. 1 illustrates the measurement of the cytotoxicity of the planarian aqueous extract on human lymphocyte cells.

FIGS. 2A to 2D illustrate the measurement of the planarian aqueous extract on the multiplication of human stem cells. Only a dilution comprised (1/20, B) between 1/5 (A) and 1/100 (C) allows a significant induction (*p<0.05) of the proliferation of human stem cells compared to untreated cells. FIG. 2D shows the growth of human stem cells treated with the planarian aqueous extract diluted to 1/20 for 9 days and the control without treatment. The cells are stained with Giemsa.

FIGS. 3A to 3D illustrate the measurement of the effect of the planarian aqueous extract on the multiplication of human epithelial cells. The cells used are human epithelial cells (ATCC CCL-2). A dilution (1/20; B) and 1:5 (A) and 1:100 (C) allows a significant induction (*p<0.05) of the proliferation of human epithelial cells compared to untreated cells. FIG. 3D shows the growth of human epithelial cells treated with the planarian aqueous extract diluted to 1/20 for 9 days and the control without treatment. The cells are stained with Giemsa.

FIGS. 4A to 4C illustrate the measurement of the effect of the planarian aqueous extract on the proliferation of human endothelial cells. The cells used are human endothelial cells (ATCC CRL-1730). A dilution (1/20; B) and 1:5 (A) and 1:100 (C) allows a significant induction (*p<0.05) of the proliferation of human endothelial cells compared to untreated cells.

FIGS. 5A to 5C illustrate the measurement of the effect of the aqueous extract of heat-treated planarian on the replication of human stem cells (ATCC CLS-300702), human endothelial cells (ATCC CRL-1730), human epithelial cells (ATCC CCL-2). A 1/20 dilution of the planarian aqueous extract is treated at 95° C. for 10 minutes is added to the human cells of interest and the multiplication of the cells is measured after 9 days. The heat inactivates the activity of the planarian aqueous extract since no cell proliferation is observed either for human stem cells (A), human epithelial cells (B), or human endothelial cells (C). The aqueous heat-inactivated planarian extract does not induce cell multiplication.

FIGS. 6A and 6B illustrate the measurement of the effect of the planarian aqueous extract on the healing of a cellular layer of human stem cells and human epithelial cells. The human cells of interest are cultured to confluence until a cellular layer is formed. A 325 μm wide lesion of the cellular layer is made using a calibrated needle, then the planarian aqueous extract diluted to 1/20^{th} is added or not to the cell culture. Healing of the cellular layer (complete filling of the lesion with new cells) is monitored over time. It can be seen that for human stem cells, as for epithelial cells, the healing of the lesion of the cellular layer is almost complete after 24 hours, and the lesion is completely closed after 48 hours of treatment with the planarian aqueous extract diluted to 1/20^{th}. In the absence of treatment, healing is not complete after 48 hours.

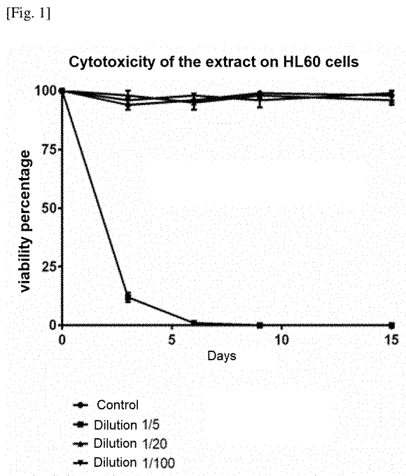

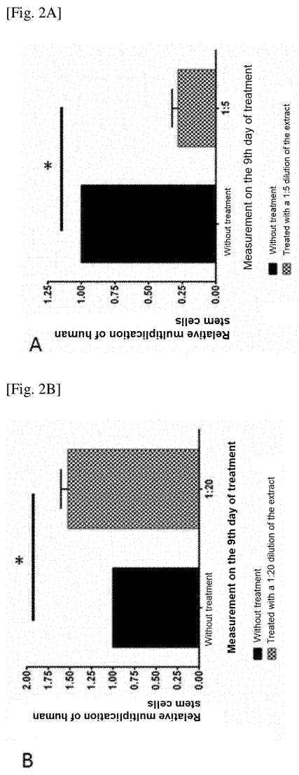

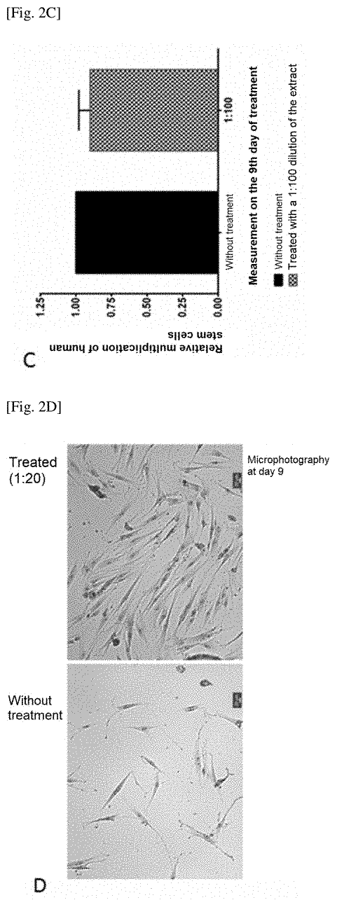

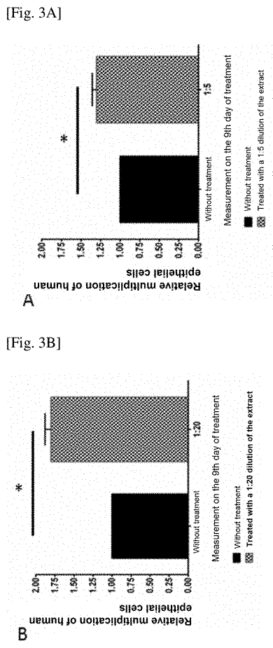

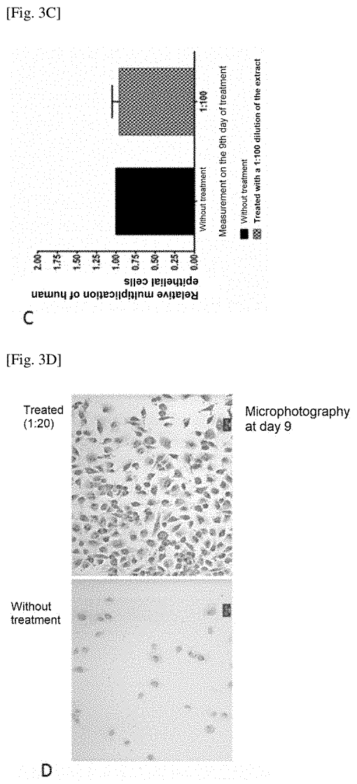

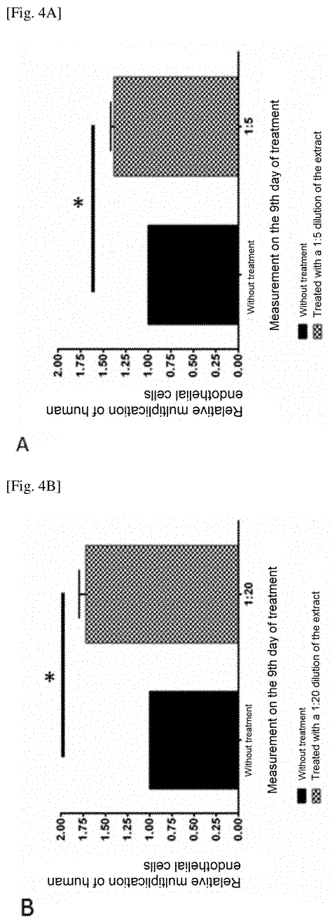

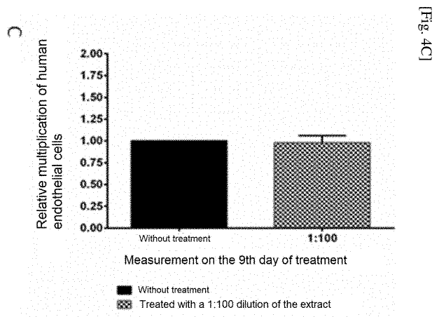

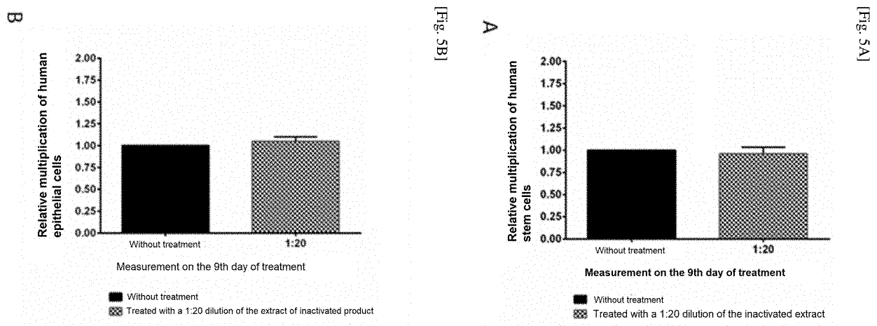

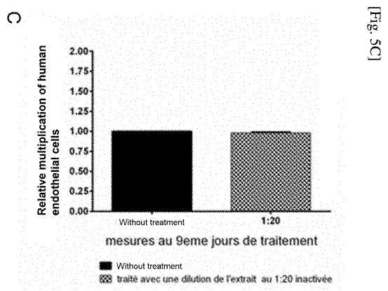

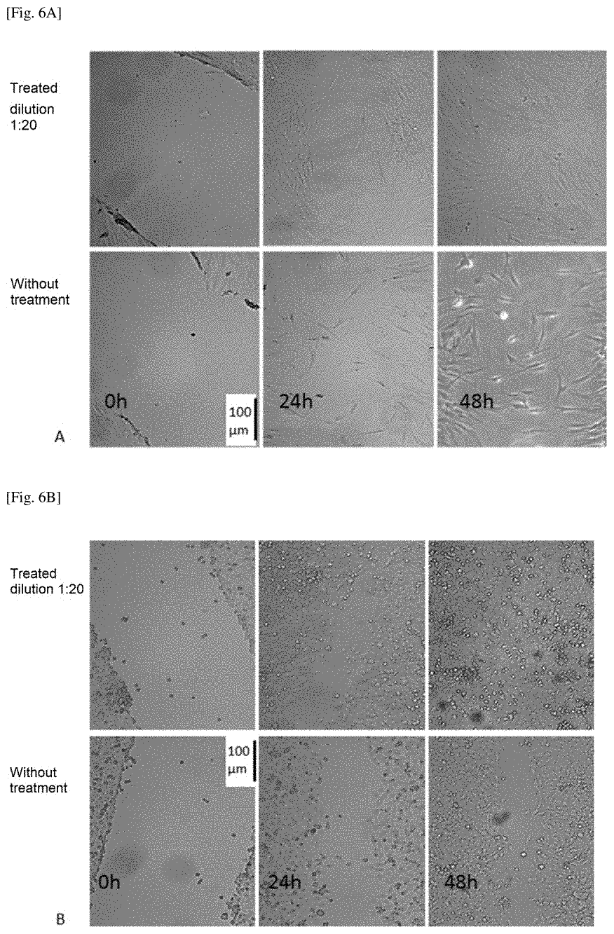

1. A method for feeding flatworms, in which the flatworms are fed exclusively with malted yeast.

2. A method for preparing a flatworm extract, comprising the steps of:
(i) feeding flatworms by implementing the method of claim 1, and
(ii) preparing a flatworm extract from the flatworms obtained in step (i).

3. A flatworm extract fed exclusively with malted yeast capable of being obtained by implementing the method according to claim 2, said flatworms being free of human-pathogenic microorganisms.

4. The extract according to claim 3, said human-pathogenic microorganisms are Aeromonas veronii, Staphylococcus capitis, Pseudomonas fluorescens, Acinetobacter guillouiae, Aeromonas hydrophila, Delftia acidovorans, Comamonas testosteroni, Sphingomonas paucimobilis, Staphylococcus epidermidis, Micrococcus luteus and Staphylococcus haemolyticus The extract according to claim 3, said human-pathogenic microorganisms are Aeromonas veronii, Staphylococcus capitis, Pseudomonas fluorescens, Acinetobacter guillouiae, Aeromonas hydrophila, Delftia acidovorans, Comamonas testosteroni, Sphingomonas paucimobilis, Staphylococcus epidermidis, Micrococcus luteus and Staphylococcus haemolyticus

5. The extract according to claim 3, the flatworm being of the class of Rhabditophora, preferably being a planarian, preferably belonging to the Schmidtea mediterranea species.

6. The extract according to claim 5, being freed from any insoluble solid debris.

7. The extract according to claim 3, comprising from 15 μg to 50 mg of protein per mL of extract (mg/mL).

8. The extract according to claim 3, comprising less than 0.5 mg/mL of protein per ml of extract (mg/mL), for example between 15 μg/mL and 0.3 mg/mL, preferably from 15 μg/mL to 75 μg/mL.

9. The extract according to claim 3, having a MALDI-TOF spectrum corresponding to the data shown in Table 1.

10. A composition comprising one or more flatworms fed with malted yeast, said flatworm(s) being free of human-pathogenic microorganisms.

11. The composition comprising an extract according to claim 3.

12. A method for eliminating pathogenic microorganisms present in one or more flatworms, said method comprising a step which consists in feeding one or more flatworms exclusively with malted yeast for a time sufficient to eliminate said pathogenic microorganisms.

13. A method for preparing an extract according to claim 3, comprising the steps of:
(a) obtaining flatworms free of human-pathogenic microorganisms by feeding one or more flatworms exclusively with malted yeast for a time sufficient to eliminate said pathogenic microorganisms;
(b) preparing a flatworm extract from the flatworms obtained in step (a).

14. A use of an extract according to claim 3 of a composition comprising the extract, for cultivating human or animal cells in vitro or ex vivo, in which said human cells are not human embryonic stem cells requiring the destruction of a human embryo and having the capacity to develop into a human being.

15. The extract according to claim 3 or composition comprising the extract for the use thereof as a medicament.

16. The extract according to claim 3 or composition comprising the extract for the use thereof to promote the healing of a skin wound or a skin burn.
